(19) United States
(12) Patent Application Publication
(10) Pub. No.: US 2024/0218308 A1
(43) Pub. Date: Jul. 4, 2024

(54) CELL CULTURE FLASK

(71) Applicant: LIFE TECHNOLOGIES CORPORATION, Carlsbad, CA (US)

(72) Inventors: Samantha STEPNOWSKI, Frederick, MD (US); Jonathan ZMUDA, Frederick, MD (US); Keith WHITTLINGER, Penfield, NY (US); Babak HAMIDIAN, Rockville, MD (US)

(21) Appl. No.: 18/399,270

(22) Filed: Dec. 28, 2023

Related U.S. Application Data
(63) Continuation-in-part of application No. 29/869,448, filed on Dec. 30, 2022.
(60) Provisional application No. 63/613,402, filed on Dec. 21, 2023.
(60) Provisional application No. 63/494,656, filed on Apr. 6, 2023.
(60) Provisional application No. 63/478,003, filed on Dec. 30, 2022.

(51) Int. Cl. C12M 1/24

(52) U.S. Cl. CPC C12M 23/08

(57) ABSTRACT

A cell culture flask having a flask body that defines a cavity for receiving a cell culture medium is provided. The flask body includes an elongate neck that defines an opening to the cavity, a base, and a sidewall that extends from the elongate neck to the base of the flask body. The sidewall includes a circumferential beveled portion, which may or may not be faceted, that extends from an outermost periphery of the flask body to the base of the flask body at a constant angle relative to a central axis of the flask to define a sidewall angle.

### CROSS-REFERENCE TO RELATED APPLICATIONS

This application claims the benefit of priority under 35 U.S.C. § 119(e) to U.S. Provisional Application No. 63/613,402 filed on Dec. 21, 2023, to U.S. Provisional Application No. 63/494,656 filed on Apr. 6, 2023 and to U.S. Provisional Application No. 63/478,003 filed on Dec. 30, 2022, and this application is a continuation-in part of U.S. application Ser. No. 29/869,448 filed on Dec. 30, 2022, the contents of which are hereby expressly incorporated herein by reference in their entirety as though fully set forth herein.

### TECHNICAL FIELD

This invention relates generally to laboratory ware and, more particularly, to a cell culture flask.

### BACKGROUND

Shaker flasks, otherwise referred to as cell culture flasks, are commonly used in a laboratory setting to cultivate biological organisms. In this regard, a quantity of biological organisms, such as any one of prokaryotic, mammalian, yeast, or insect cells, for example, are placed in a cell culture flask containing a cell culture medium to thereby grow the biological organisms into significantly more organisms as quickly as possible. The growth of the biological organisms, or cells, is dependent on exposure of the cell culture medium and thus the biological organisms to adequate levels of oxygen. That is, cell growth increases with aeration of the cell culture medium, resulting in a continuous exchange of oxygen and removal of carbon dioxide therefrom. To improve aeration and the transfer of oxygen into the cell culture medium (i.e., the oxygen transfer rate (“OTR”)), the shaker flask is typically agitated using a laboratory shaker. To this end, shaking of the flask creates a vortex that exposes more liquid surface to the oxygen in the headspace of the flask.

Generally, oxygen transfer takes place via two liquid surfaces within the shaker flask-a bulk liquid surface of the cell culture medium and a liquid film that forms on the wetted shaker flask sidewalls. In this regard, the OTR in shaker flasks is determined by the flask size and geometry, agitation speed, fill volume, and ambient conditions. With respect to flask geometry, baffles can be used to create turbulent flow to improve aeration and oxygen transfer during agitation. Further, increasing the shaking speed can also increase aeration of the cell culture medium and thus oxygen transfer. However, while baffles and agitation speed can improve aeration during shaking operations, baffles in particular can cause cells to experience higher levels of shear stress (e.g., hydrodynamic stress) which can be detrimental to cell viability. Moreover, aggressive baffles can cause foaming of the cell culture medium which hinders oxygen transfer.

Appropriate agitation during cell culturing and growth is extremely important for cell viability and needs to be balanced with cell suspension homogeneity, particularly for cell types sensitive to shear stress such as mammalian and insect cells, for example. To this end, sharp features inside a shaker flask, such as steep baffles with sharp edges, can easily stress and damage cells over a range of various shaker speeds. Therefore, a need exists to provide a cell shaker flask capable of aerating a cell culture medium to quickly and effectively culture biological organisms, especially those sensitive to shear stress, without the adverse effects of shear stress and foaming.

### SUMMARY

The present invention overcomes the foregoing and other shortcomings and drawbacks of cell culture flasks. While the present invention will be discussed in connection with certain embodiments, it will be understood that the present invention is not limited to the specific embodiments described herein.

Accordingly, in one aspect, provided herein is a cell culture flask, having a flask body defining a cavity for receiving a cell culture medium. In one embodiment, the flask body includes an elongate neck that defines an opening to the cavity; a base; a sidewall that extends from the elongate neck to the base of the flask body, the sidewall including a circumferential beveled portion that extends from an outermost periphery of the flask body to the base of the flask body at a constant angle relative to a central axis of the flask to define a sidewall angle.

In certain embodiments, the flask having the circumferential beveled portion also includes at least one baffle formed in the flask body so as to extend in a radially inward direction relative to the central axis of the flask to form an indent in the flask. In some embodiments, the at least one baffle includes six baffles spaced equidistantly apart and distributed circumferentially about the flask body. In other embodiments, the at least one baffle includes a first pair of baffles spaced 60° apart from each other about the central axis of the flask and a second pair of baffles spaced 60° apart from each other about the central axis of the flask, wherein the first pair of baffles are diametrically opposed from the second pair of baffles about the central axis of the flask.

In another embodiment, the flask body includes an elongate neck that defines an opening to the cavity; a base; a sidewall that extends from the elongate neck to the base of the flask body, the sidewall including a circumferential faceted portion that extends from an outermost periphery of the flask body to the base of the flask body that is defined by a plurality of facets distributed circumferentially about the flask body.

In certain embodiments, the flask having the circumferential faceted portion also includes at least one baffle formed in surfaces of one facet of the plurality of facets and the base of the flask body so as to extend in a radially inward direction relative to a central axis of the flask. In some embodiments, the at least one baffle includes six baffles spaced equidistantly apart and distributed circumferentially about the flask body with each baffle being formed on surfaces of a different one of the plurality of facets and the base. In other embodiments, the at least one baffle includes a first pair of baffles spaced 60° apart from each other about the central axis of the flask so as to be formed on surfaces of a different one of the plurality of facets and the base, and a second pair of baffles spaced 60° apart from each other about the central axis of the flask so as to be formed on surfaces of a different one of the plurality of facets and the base, wherein the first pair of baffles are diametrically opposed from the second pair of baffles about the central axis of the flask.

In some embodiments, provided is, in combination the cell culture flask a described herein and a laboratory shaker.

In another aspect, also provided is method of culturing cells, that includes providing a cell culture flask as described herein, introducing a cell culture medium and one or more cells into the cavity; and culturing the cells under conditions to support growth and/or expansion of the one or more cells.

In another embodiment, provided are methods of expressing a protein of interest, that includes providing a cell culture flask as described herein, introducing a cell culture medium and one or more cells configured to express the protein of interest into the cavity; and culturing the cells under conditions to support expression of the protein of interest. In some embodiments, the method further comprises isolating the protein of interest from the cell culture medium and/or the one or more cells.

In another embodiment, provided are methods of producing a virus vector, that includes providing a cell culture flask as described herein, introducing a cell culture medium and one or more cells configured to express the virus vector into the cavity; and culturing the cells under conditions to support expression of the virus vector. In some embodiments, the method further comprises isolating the virus vector from the cell culture medium and/or the one or more cells.

Various additional features and advantages of the invention will become more apparent to those of ordinary skill in the art upon review of the following detailed description of one or more illustrative embodiments taken in conjunction with the accompanying drawings.

### BRIEF DESCRIPTION OF THE DRAWINGS

The accompanying drawings, which are incorporated in and constitute a part of this specification, illustrate one or more embodiments of the invention and, together with the general description given above and the detailed description given below, serve to describe the one or more embodiments of the invention.

FIG. 1 is a front plan view of a cell culture flask according to one embodiment of the present invention.

FIG. 2 is a cross-sectional view of the shaker flask taken along line 2-2 in FIG. 1.

FIG. 3 is an enlarged cross-sectional view of the base of the shaker flask of FIGS. 1-2, illustrating details of a small baffle.

FIG. 4 is a bottom perspective view of the base of the shaker flask of FIGS. 1-3, illustrating the base isolated from the shaker flask.

FIG. 5 is a partial bottom view of the base of the shaker flask of FIG. 4, illustrating details of a small baffle.

FIG. 6 is a bottom plan view of the base of the shaker flask of FIGS. 1-5.

FIG. 7A is a view similar to FIG. 6, illustrating a first and a second pair of baffles.

FIG. 7B is a bottom plan view of a base of a shaker flask having small baffles in accordance with an embodiment of the present invention.

FIGS. 8A-8C are bottom plan views of embodiments of a base of a shaker flask having large baffles in accordance with embodiments of the present invention.

FIG. 9 is a front plan view of a cell culture flask in accordance with another embodiment of the present invention.

FIG. 10 is a cross-sectional view of the shaker flask taken along line 10-10 in FIG. 9.

FIG. 11 is an enlarged cross-sectional view of the base of the shaker flask of FIGS. 9-10, illustrating details of a facet.

FIG. 12 is a bottom perspective view of the base of the shaker flask of FIGS. 9-11, illustrating the base isolated from the shaker flask.

FIG. 13 is a partial bottom plan view of the base of the shaker flask of FIG. 12, illustrating details of the facets.

FIG. 14 is a bottom plan view of the base of the shaker flask of FIG. 12.

FIG. 15 is a front plan view of a cell culture flask in accordance with another embodiment of the present invention.

FIG. 16 is a cross-sectional view of the shaker flask taken along line 16-16 in FIG. 15.

FIG. 17 is an enlarged cross-sectional view of the base of the shaker flask of FIGS. 15-16, illustrating details of a small baffle.

FIG. 18 is a bottom perspective view of the base of the shaker flask of FIGS. 15-17, illustrating the base isolated from the shaker flask.

FIG. 19 is a partial bottom plan view of the base of the shaker flask of FIG. 18, illustrating details of a small baffle.

FIG. 20 is a bottom plan view of the base of the shaker flask of FIG. 15.

FIG. 21A is a view similar to FIG. 20, illustrating a first and a second pair of baffles.

FIG. 21B is a bottom plan view of a base of a shaker flask having small baffles in accordance with an embodiment of the present invention.

FIGS. 22A-22C are bottom plan views of embodiments of a base of a shaker flask having large baffles in accordance with embodiments of the present invention.

FIG. 23 is a diagrammatic view showing a cell culture flask positioned on a laboratory shaker within an incubator.

FIG. 24 is a front plan view of a cell culture flask according to one embodiment of the present invention.

FIG. 25 is a cross-sectional view of the shaker flask taken along line 24-24 in FIG. 1.

FIG. 26 is an enlarged cross-sectional view of the base of the shaker flask of FIGS. 24-25, illustrating details of the beveled portion.

FIG. 27 is a bottom perspective view of the base of the shaker flask of FIGS. 24-26, illustrating the base isolated from the shaker flask.

FIG. 28 is a partial bottom view of the base of the shaker flask of FIG. 27.

FIG. 29 is a bottom plan view of the base of the shaker flask of FIGS. 24-28.

FIG. 30 is a graph depicting growth performance of Expi293 cells cultured in faceted-bottomed flasks at varying end volumes and control 30 mL cultures.

FIG. 31 is a graph depicting growth performance of ExpiSf9 cells cultured in an beveled-bottom flask and a faceted-bottom flask at a 2-liter end volume.

FIG. 32 is a graph depicting growth performance of ExpiCHO cells cultured in a beveled-bottom flask at a 2-liter end volume and control 30 mL volume.

FIG. 33 is a bar graph depicting antibody expression levels from transfected Expi293 cells cultured at a 2.25-liter end volume and control 30 mL volume.

FIG. 34 is a bar graph depicting GFP expression levels from baculovirus infected Expi293 cells cultured at a 2-liter end volume and control 30 mL volume.

FIG. 35 is a bar graph depicting protein expression levels from transfected ExpiCHO cells cultured at a 2-liter end volume and control 30 mL volume following the Max Titer protocol for expression.

FIG. 36 is a bar graph depicting protein expression levels from transfected ExpiCHO cells cultured at a 2-liter end volume and control 30 mL volume following the Standard protocol for expression.

### DETAILED DESCRIPTION

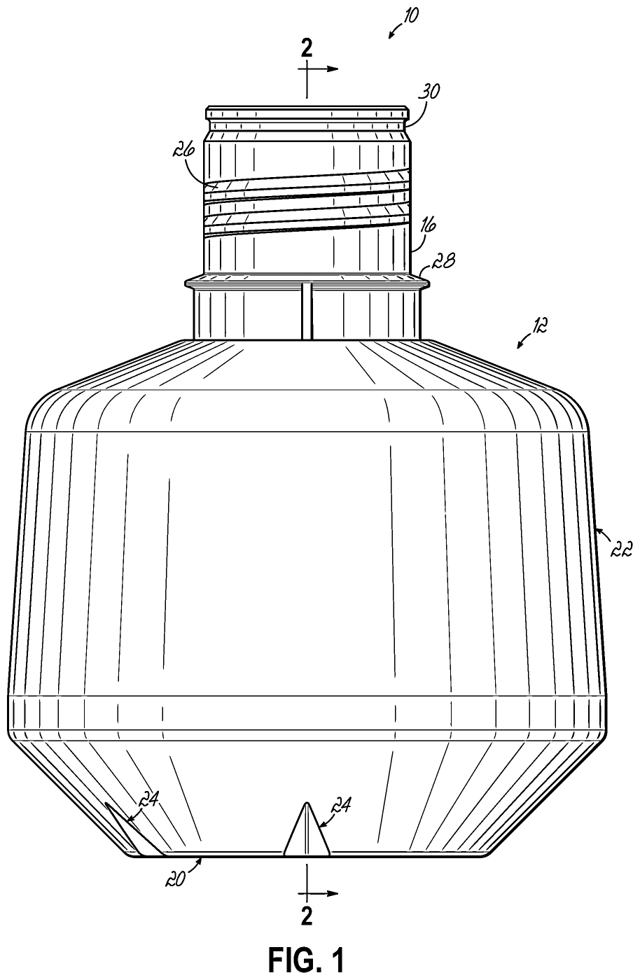

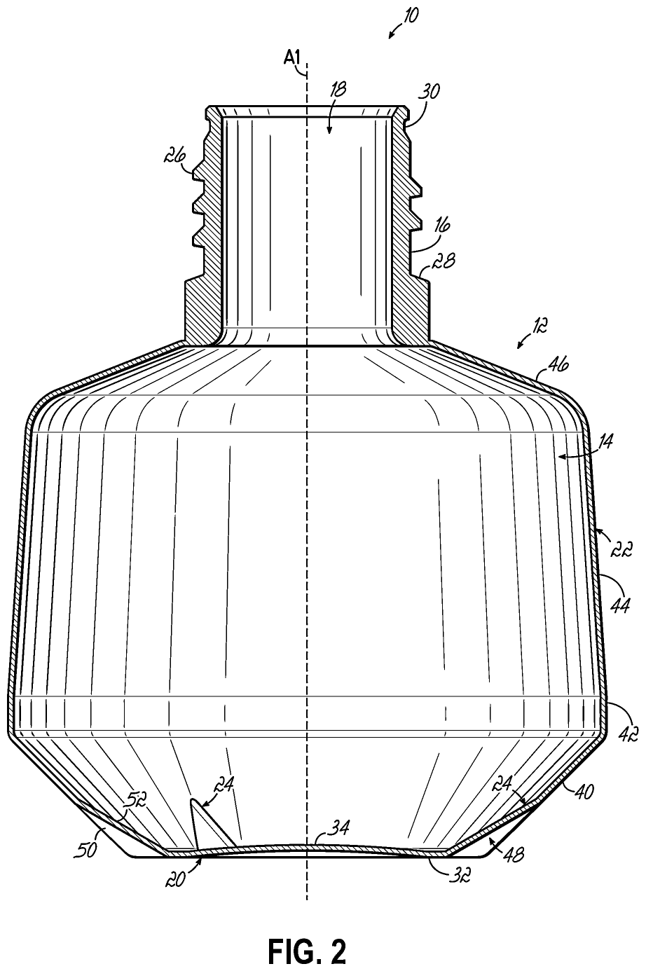

Referring now to the figures, and in particular to FIGS. 1 and 2, a cell culture flask 10 in accordance with a first embodiment of the present invention is shown. The cell culture flask 10 includes a flask body 12 that defines a cavity 14 for receiving a cell culture medium for culturing one or more biological organisms (i.e., cells). As will be described in further detail below, the cell culture flask 10 is configured to be placed on a laboratory shaker and agitated at various agitation frequencies and movements to express a protein of interest into the cavity 14. The flask body 12 includes an elongate neck 16 that defines an opening 18 to the cavity 14, a base 20, and a sidewall 22 that extends from the elongate neck 16 to the base 20. The cell culture flask 10 further includes at least one baffle 24 formed in the flask body 12 that is configured to increase oxygen exchange during agitation of the cell culture medium contained in the cell culture flask 10, particularly for cell types sensitive to shear stress, to thereby increase cell titer and protein expression while minimizing cell shear and foaming, as will be described in further detail below.

The exemplary cell culture flask 10 may be a 5-liter (L) flask, by way of example, having a minimum of a 5 L fill volume below the elongated neck 16. The cell culture flask 10 may have a total cavity 14 fill volume of 5.5 L, for example. However, the preferred working volume of the cell culture flask 10 may be within the range of between 2.5 L to 3.5 L. In either case, embodiments of the invention can also be applied to the design and manufacture of lower or higher volume cell culture flasks (e.g., 10 L), and particularly to flasks having a fill volume within the range of between 125 mL to 28000 mL, for example. To this end, the drawings are not intended to be limiting.

With continued reference to FIGS. 1 and 2, the elongate neck 16 of the flask body 12 defines the opening 18 to the cavity 14 through which cell culture medium and cells may be introduced into, and removed from, the cell culture flask 10. The elongate neck 16 includes a threaded portion 26 configured to threadably receive a closure device (not shown) and a collar 28 that defines an abutment surface for the closure device. The elongate neck 16 may further include a groove 30 configured to receive an O-ring (not shown) used to form a seal between the elongate neck 16 and the closure device. The closure device is configured to close the opening 18 to prevent spilling of the cell culture medium held in the cell culture flask 10, for example. The closure device may be a vented cap to allow air exchange, such as oxygen to enter the cavity and carbon dioxide to leave the cavity. To this end, the threaded portion 26 may include size 83B threads configured to receive an 83B threaded closure device, for example.

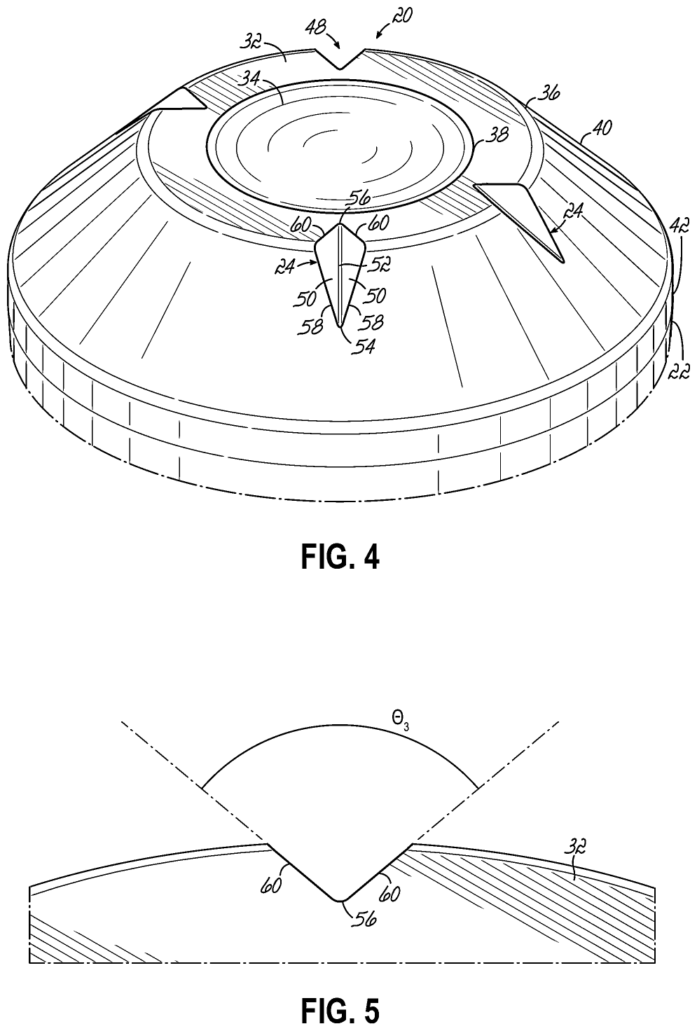

With reference to FIGS. 2 and 4, the base 20 of the of the flask body 12 includes a base surface 32 and a central concave portion 34 that is curved gently upward and into the cavity 14 from the base surface 32. The profile of the central concave portion 34 serves to direct the vortex of cell culture medium over the at least one baffle 24 during agitation of the cell culture flask 10. As shown in FIG. 4, the base surface 32 comprises a generally flat annular disk having an outer circumferential edge 36 that defines an outer diameter of the base surface 32 and an inner circumferential edge 38 that defines an inner diameter of the base surface 32. To this end, the base surface 32 is configured to support the cell culture flask 10 in an upright position on a support surface, such as a laboratory shaker, for example.

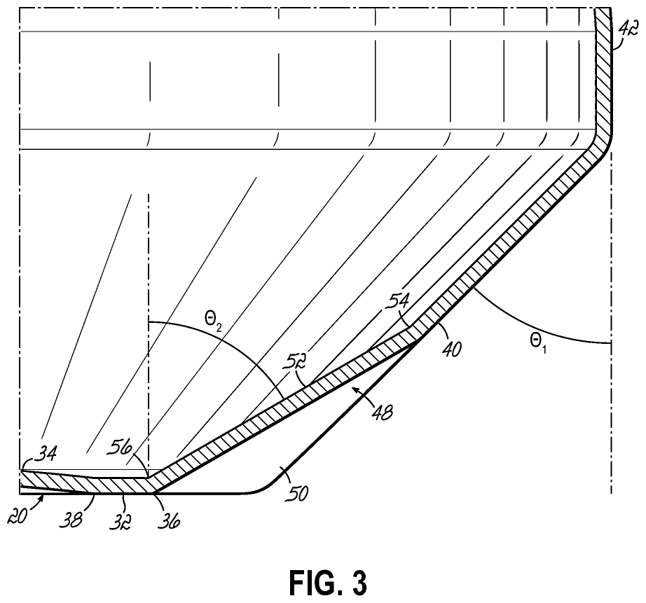

Referring now to FIGS. 1-3, the sidewall of the flask body 12 includes a first circumferential beveled portion 40 that extends upward from the base 20 of the flask body 12 to an outermost periphery 42 of the flask body 12 in a radially outward direction relative to an central axis A1 of the cell culture flask 10. The outermost periphery 42 of the cell culture flask 10 is defined as a portion of the flask 10 where a diameter of the flask body 12 is the greatest. In any event, the first beveled portion 40 extends radially outwardly and upwardly at a constant angle relative to the central axis A1 (i.e., a vertical axis) of the cell culture flask 10 to define a sidewall angle θ₁, as shown in FIG. 3. The sidewall angle θ₁ may be within a range of between 30° to 60°, for example. In a preferred embodiment, the sidewall angle θ₁ is 45°.

The sidewall 22 of the flask body 12 further includes a second circumferential beveled portion 44 that extends from the outermost periphery 42 of the flask body 12 to a shoulder 46 of the flask body 12. The shoulder 46 and the second beveled portion 44 of the sidewall 22 may be generally symmetrical about the central axis A1 of the cell culture flask 10. As shown in FIG. 2, the second beveled portion 44 extends from the outermost periphery 42 of the flask body 12 to the shoulder 46 in a radially inward direction relative to the central axis A1 of the cell culture flask 10. In particular, the second beveled portion 44 may extend at an angle of between 2° to 5° from vertical toward the central axis A1 of the cell culture flask 10. In the exemplary embodiment of the cell culture flask 10 shown, the second beveled portion 44 extends radially inwardly at an angle of 3.8° from vertical. The shoulder 46 extends upwardly from the second beveled portion 44 to the elongated neck 16 at an angle of between 15° to 20° from horizontal (i.e., a horizontal plane transverse to the central axis A1 of the cell culture flask 10). In the exemplary embodiment of the cell culture flask 10 shown, the shoulder 46 extends from the second beveled portion 44 to the elongate neck 16 at an angle of 16.164° from horizontal.

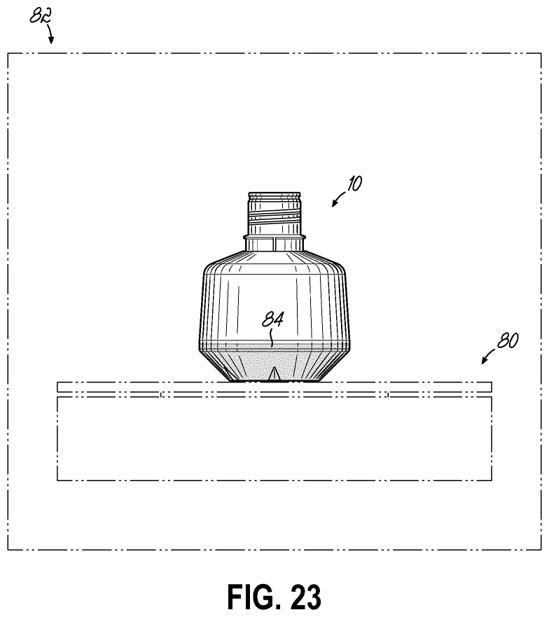

The overall height of the cell culture flask 10 (i.e., a distance measured between the base 20 of the flask body 12 and an apex of the elongate neck 16) may be 10.8 inches. That way, the cell culture flask 10 effectively fits within a standard tabletop incubator having a laboratory shaker and a cover or hood (e.g., FIG. 23), yet provides clearance between the opening 18 in the elongate neck 16 and the cover for a pipette or other tools used to add/remove fluids, such as cell culture medium and cells, to/from the cell culture flask 10. To this end, the elongate neck 16 may have an inner diameter (ID) of 2.57 inches and a height of 3.3 inches to accommodate 50 mL serological pipettes, for example.

With reference to FIGS. 2-5, the cell culture flask 10 further includes at least one baffle 24 formed in the flask body 12. In the exemplary embodiment shown, the cell culture flask 10 includes four baffles symmetrically spaced apart about the central axis A1 of the cell culture flask 10. As shown in FIGS. 2 and 3, each baffle 24 is formed in the flask body 12 so as to extend in a radially inwardly direction relative to the central axis A1 of the cell culture flask 10 to form an indent 48 in the flask body 12. In particular, each baffle 24 is generally V-shaped to define a pair of flattened sidewalls 50 that join together along an edge 52 which defines a peak for each baffle 24 within the cavity 14 of the flask body 12 (e.g., FIG. 2). As shown in FIG. 5, each baffle 24 is generally triangular in transverse cross-sectional shape with the edge 52 forming an apex of the triangle. Notably, the edge 52 is curved in transverse cross-section (i.e., radiused) to facilitate a smooth transfer of cell culture medium over the baffle 24 to minimize shear stress and cell damage during agitation of the cell culture medium.

As shown in FIGS. 2 and 3, the pair of sidewalls 50 of each baffle 24 extend between the first beveled portion 40 of the sidewall 22 and the base 20 of the flask body 12 such that the edge 52 extends in a radially inward direction relative to the central axis A1 of the cell culture flask 10. In that regard, the edge 52 extends from a first terminal end 54 located on the first beveled portion 40 of the sidewall 22 and a second terminal end 56 located on the base surface 32 of the base 20 of the flask body 12. More particularly, as shown in FIG. 3, the edge 52 of each baffle is angled relative to the central axis A1 (i.e., a vertical axis) of the cell culture flask 10 to define a baffle angle θ₂. The baffle angle θ₂ may be within a range of between 45° to 70° (or 20° to 45° from a horizontal base plane), for example. In a preferred embodiment, the baffle angle θ₂ is 60° (or 30° from a horizontal base plane).

Referring now to FIGS. 3-5, each baffle sidewall 50 is generally triangular in shape to define a first leg edge 58 and a second leg edge 60 with a hypotenuse being the common edge 52 shared by the pair of sidewalls 50. Each first leg edge 58 extends along the first beveled portion 40 of the sidewall 22 and between the first terminal end 54 of the edge 52 and the outer edge 36 of the base surface 32. Each second leg edge 60 extends along the base surface 32 and between the outer edge 36 of the base surface 32 and the second terminal end 56 of the edge 52. As best shown in FIGS. 4 and 5, each baffle 24 is generally V-shaped in transverse cross-section as a result of the angled relationship between the pair of sidewalls 50. More particularly, as shown in FIG. 5, the pair of sidewalls 50 are angled relative to each other to form a baffle sidewall angle θ₃ therebetween. The baffle sidewall angle θ₃ may be within a range of between 90° to 110°, for example. In a preferred embodiment, the baffle sidewall angle θ₃ is 100°. To this end, it is important to have a wide baffle sidewall angle θ₃ (i.e., 100°) minimize the steepness of the baffle to thereby reduce shear stress and cell damage during agitation of the cell culture medium.

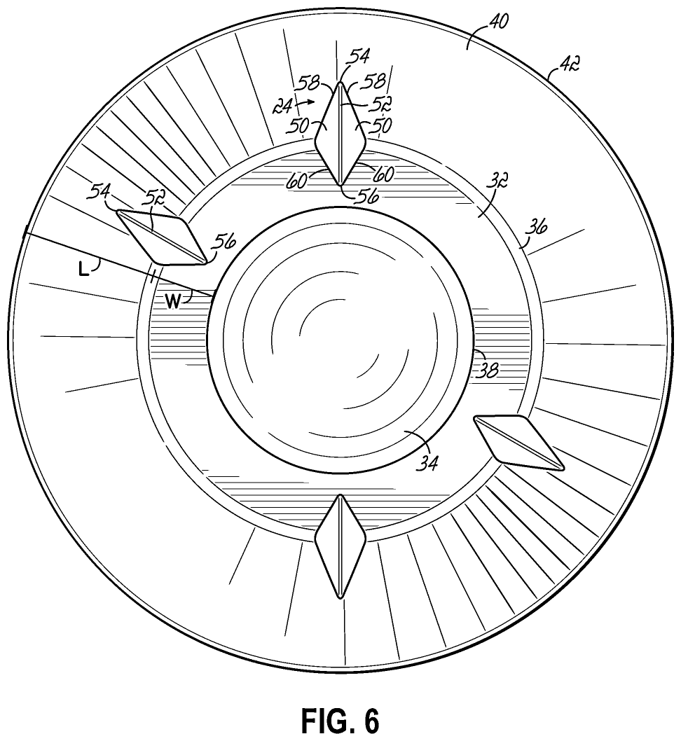

As best shown in FIGS. 4 and 6, each baffle 24 extends along the first beveled portion 40 of the flask body 12 a greater distance compared to the base surface 32. As a result, a length of each first leg edge 58 is greater than a length of each second leg edge 60. In that regard, the first beveled portion 40 of the sidewall 22 includes a length L measured as a distance between the outer edge 36 of the base surface 32 and the outermost periphery 42 of the flask body 12. Each first leg edge 58 may extend a distance from the outer edge 36 of the base surface 32 to locate the first terminal end 54 of the edge 52 within an area on the first beveled portion 40 of the sidewall 22 that is between 40% to 60%, and preferably 45% to 50%, of the length L of the circumferential beveled portion 40 of the sidewall 22 measured from the outer edge 36 of the base surface 32 of the flask body 12. To this end, the location of the first terminal end 54 of the edge 52 along the first beveled portion 40 of the sidewall 22 influences the baffle angle θ₂.

With continued reference to FIGS. 4 and 6, the base surface 32 includes a width W measured as a distance between the inner edge 38 and the outer edge 36 of the base surface 32 of the flask body 12. In that regard, each second leg edge 60 extends a distance from the outer edge 36 of the base surface 32 to locate the second terminal end 56 of the edge 52 within an area on the base surface 32 that is between 50% to 70%, and preferably 60% to 65%, of the width W of the base surface 32 measured from the outer edge 36 of the base surface 32 of the flask body 12. To this end, the location of the second terminal end 56 of the edge 52 along the base surface 32 also influences the baffle angle θ₂.

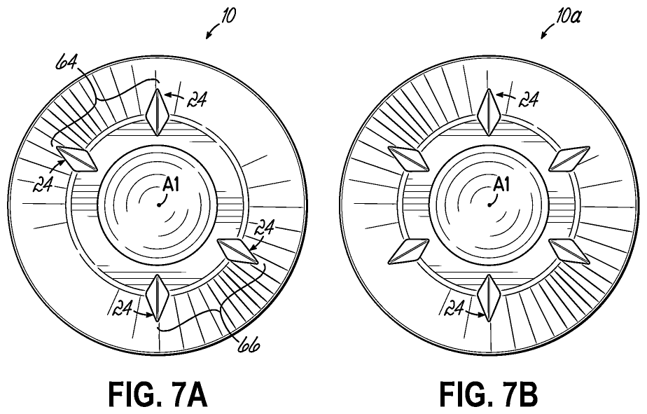

Referring now to FIG. 7A, the cell culture flask 10 includes a first pair 64, or grouping of baffles 24 and a second pair 66, or grouping of baffles 24 diametrically opposed from the first pair of baffles 64 about the central axis A1 of the cell culture flask 10. The baffles 24 that form the first pair 64 of baffles 24 are spaced 60° apart from each other about the central axis A1 of the cell culture flask 10 and the baffles 24 that form the second pair 66 of baffles 24 are also spaced 60° apart from each other about the central axis A1 of the cell culture flask 10. To this end, the baffles 24 that form the first and second pair 64, 66 of baffles 24 may be spaced further or closer apart from each other about the central axis A1 of the cell culture flask 10, such as within a range of between 50° to 70° apart, for example.

FIG. 7B illustrates a cell culture flask 10a having six baffles 24 in accordance with an embodiment of the present invention. As shown, the baffles 24 are spaced apart from each other in a symmetrical arrangement about the central axis A1 of the cell culture flask 10a with the baffles 24 being spaced equidistantly apart in 60° increments about the central axis A1 of the cell culture flask 10a. To this end, the cell culture flask 10a may include fewer or more baffles 24 spaced apart in different increments about the central axis A1 of the cell culture flask 10a, such as four baffles 24 spaced 90° apart about the central axis A1 of the cell culture flask 10a, for example.

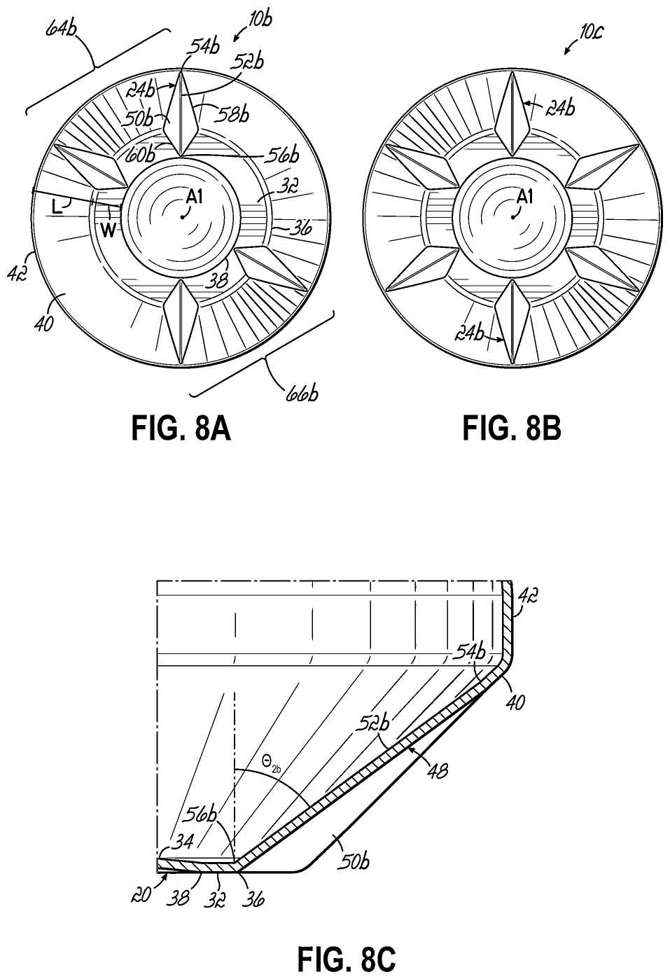

Referring now to FIGS. 8A-8C, wherein like numerals represent like features, cell culture flasks 10b, 10c in accordance with additional embodiments of the present invention are shown and will now be described. The primary differences between the cell culture flasks 10b, 10c of these embodiments and the cell culture flask 10 of the previously described embodiment is the configuration of the baffles 24b. Notably, the baffles 24b are larger compared to the baffles 24 of the previously described embodiment. The baffles 24b of FIGS. 8A-8C may be referred to as “large” baffles 24b and the baffles 24 of FIGS. 1-7B may be referred to as “small” baffles 24. In that regard, the edge 52b of each large baffle 24b is longer in length to locate the first terminal end 54b of the edge 52b a greater distance away from the outer edge 36 of the base surface 32 along the first beveled portion 40 of the flask body 12. As shown, each first leg edge 58b may extend a distance from the outer edge 36 of the base surface 32 to locate the first terminal end 54b of the edge 52b within an area on the first beveled portion 40 of the sidewall that is between 85% to 99%, and preferably 95% to 99%, of the length L of the circumferential beveled portion 40 of the sidewall 22 measured from the outer edge 36 of the base surface 32 the flask body 12. Similarly, each second leg edge 60b extends a distance from the outer edge 36 of the base surface 32 to locate the second terminal end 56b of the edge 52b within an area on the base surface 32 that is between 85% to 99%, and preferably 95% to 99%, of the width W of the base surface 32 measured from the outer edge 36 of the base surface 32 of the flask body 12. As a result of the size of the larger baffles 24b, the baffle angle θ_{2b} is steeper compared to the baffle angle θ₂ of the previously described embodiment, as shown in FIG. 8C. In that regard, the baffle angle θ_{2b} is 52° (or 38° from a horizontal base plane).

FIG. 8A illustrates a cell culture flask 10b having four large baffles 24b according to an embodiment of the present invention, with the baffles 24b being spaced apart in a symmetrical arrangement about the central axis of the cell culture flask. In that regard, the cell culture flask 10b includes a first pair 64b, or grouping of large baffles 24b and a second pair 66b, or grouping of large baffles 24b diametrically opposed from the first pair 64b of baffles 24b about the central axis A1 of the cell culture flask 10b. The baffles 24b that form the first pair 64b of baffles 24b are spaced 60° apart from each other about the central axis A1 of the cell culture flask 10b and the baffles 24b that form the second pair 66b of baffles 24b are also spaced 60° apart from each other about the central axis A1 of the cell culture flask 10b. To this end, the baffles 24b that form the first and second pair 64b, 66b of baffles 24b may be spaced further or closer apart from each other about the central axis A1 of the cell culture flask 10, such as within a range of between 50° to 70° apart, for example.

FIG. 8B illustrates a cell culture flask 10c including six large baffles 24b according to an embodiment of the present invention. As shown, the baffles 24b are spaced apart from each other in a symmetrical arrangement about the central axis A1 of the cell culture flask 10c. In particular, the baffles 24b are spaced equidistantly apart in 60° increments about the central axis A1 of the cell culture flask 10c.

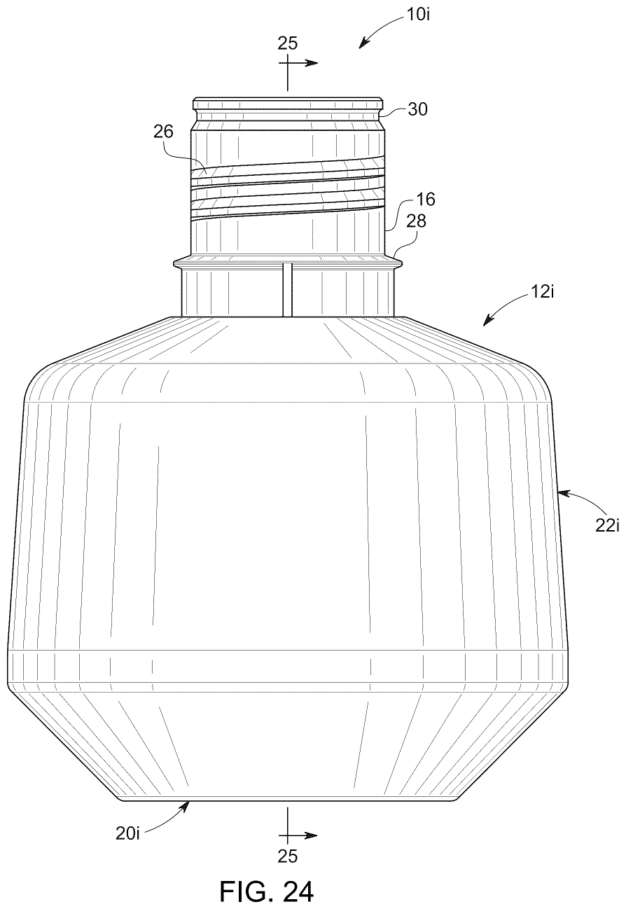

Referring now to FIGS. 24-29, wherein like numerals represent like features compared to the embodiment of the cell culture flask 10 described above with respect to FIGS. 1-6, a cell culture flask 10i in accordance with another embodiment of the present invention is shown and will now be described. The primary difference between the cell culture flask 10i of this embodiment and the cell culture flask 10 of the previously described embodiment is that the flask body 12i includes a circumferential beveled portion 40i without at least one baffle. As described in further detail below, the circumferential beveled portion 40i provides minimal turbulence during agitation of the cell culture medium contained in the cell culture flask 10i to effectively culture certain cell types sensitive to shear stress.

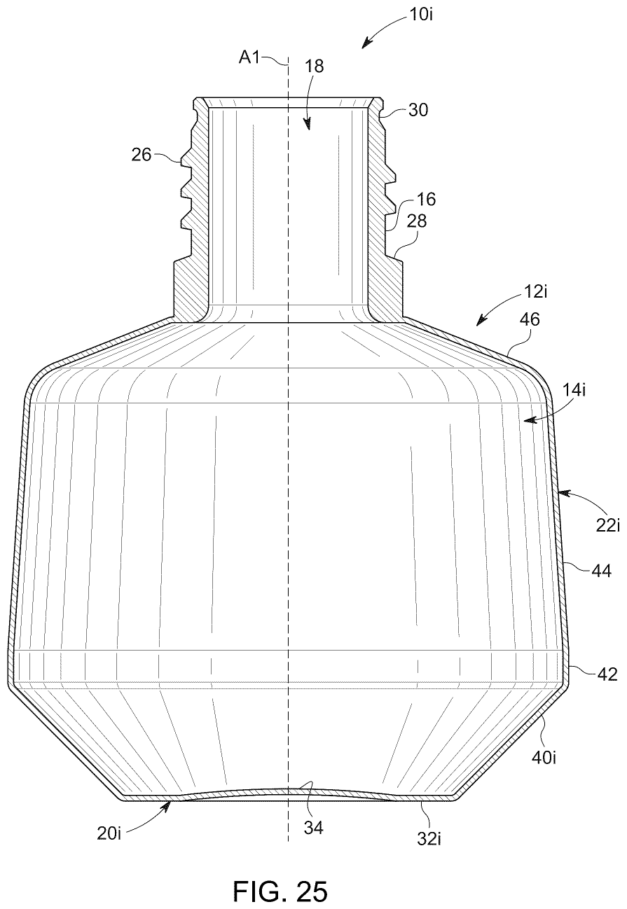

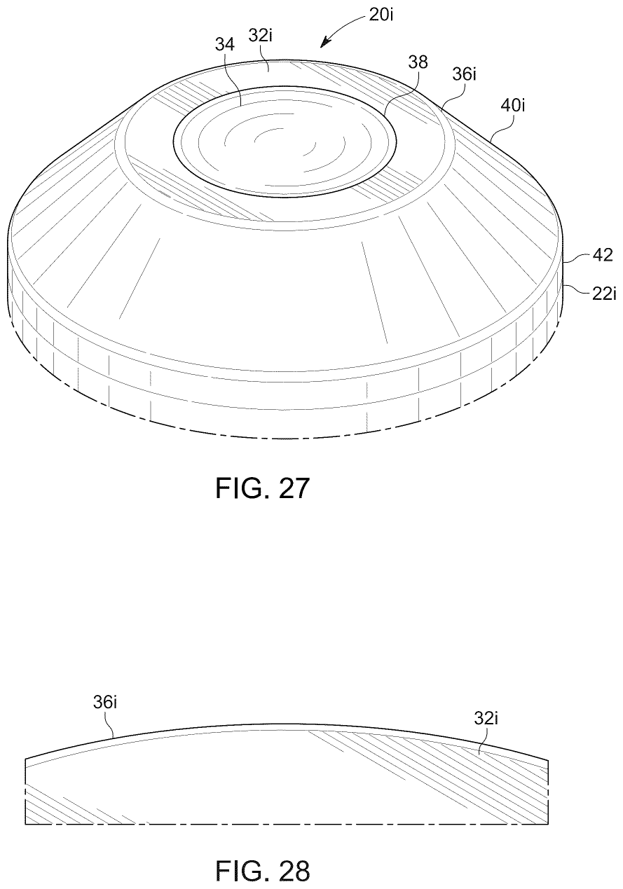

With reference to FIGS. 24 and 25, the cell culture flask 10i includes a flask body 12i that defines a cavity 14i for receiving a cell culture medium for culturing one or more cells, and an elongate neck 16 that defines an opening 18 to the cavity 14i. The flask body 12i further includes a base 20i and a sidewall 22i that extends from the elongate neck 16 to the base 20i. As best shown in FIG. 27, the base 20i of the flask body 12i includes a base surface 32i and a central concave portion 34 that is curved gently upward and into the cavity 14i from the base surface 32i. To this end, the base surface 32i comprises a generally flat annular disk having an outer circumferential edge 36i that defines an outer diameter of the base surface 32i and an inner circumferential edge 38 that defines an inner diameter of the base surface 32i. As shown in FIG. 25, the sidewall 22i includes the circumferential beveled portion 40i and a second circumferential beveled portion 44 that extends from the outermost periphery 42 of the flask body 12i to a shoulder 46 of the flask body 12i.

With reference to FIGS. 25 and 27, the base 20i of the flask body 12i includes a base surface 32 and a central concave portion 34 that is curved gently upward and into the cavity 14i from the base surface 32. The profile of the central concave portion 34 serves to create a vortex of cell culture medium during agitation of the cell culture flask 10. As shown in FIG. 27, the base surface 32 comprises a generally flat annular disk having an outer circumferential edge 36i that defines an outer diameter of the base surface 32i and an inner circumferential edge 38 that defined an inner diameter of the base surface 32. To this end, the base surface 32 is configured to support the cell culture flask 10 in an upright position on a support surface, such as a laboratory shaker, for example.

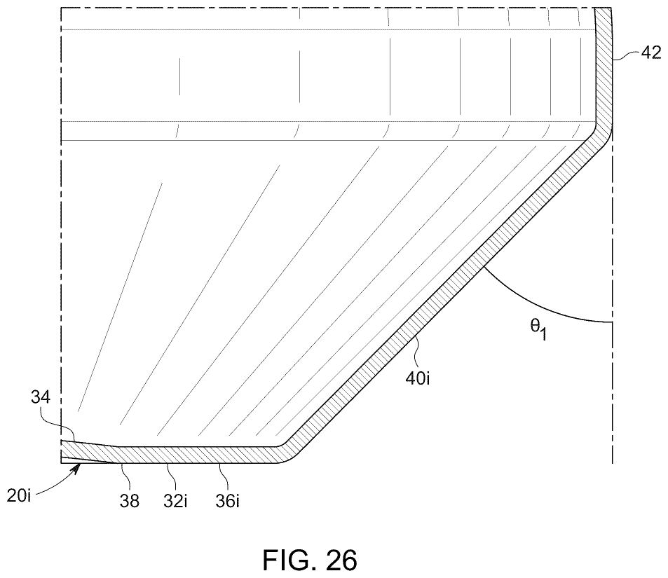

Referring now to FIGS. 24-26, the sidewall of the flask body 12i includes a first circumferential beveled portion 40i that extends upward from the base 20i of the flask body 12i to an outermost periphery 42 of the flask body 12i in a radially outward direction relative to a central axis A1 of the cell culture flask 10i. The outermost periphery 42 of the cell culture flask 10i is defined as a portion of the flask 10 where a diameter of the flask body 12i is the greatest. In any event, the first beveled portion 40i extends radially outwardly and upwardly at a constant angle relative to the central axis A1 (i.e., a vertical axis) of the cell culture flask 10i to define a sidewall angle θ₁, as shown in FIG. 26. The sidewall angle θ₁ may be within a range of between 30° to 60°, for example. In a preferred embodiment, the sidewall angle θ₁ is 45°.

With reference to FIGS. 25-28, the cell culture flask 10i does not include a baffle formed in the flask body 12. In some embodiments, the lack of baffles can be advantageous particularly for cell types sensitive to shear stress to minimize shear stress and cell damage during agitation of the cell culture medium. Compared to a cell culture flask having baffles, the beveled only cell culture flask 10i greatly reduces the risk of foaming. Use of the beveled only culture flask 10i thereby increases cell titer and protein expression while minimizing cell shear and foaming.

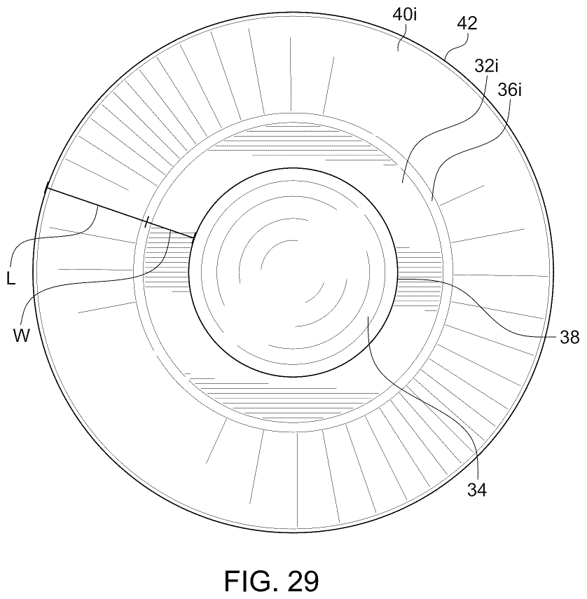

With references to FIGS. 27 and 29, the base surface 32i includes a width W measured as a distance between the inner edge 38 and the outer edge 36 of the base surface 32i of the flask body 12i.

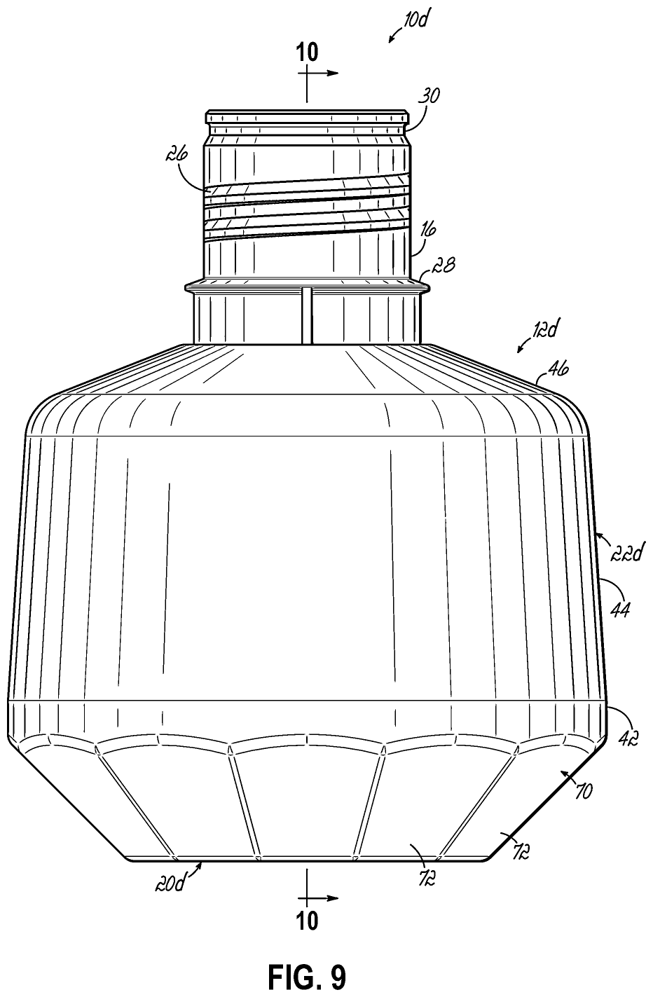

Referring now to FIGS. 9-14, wherein like numerals represent like features compared to the embodiment of the cell culture flask 10 described above with respect to FIGS. 1-7A, a cell culture flask 10d in accordance with another embodiment of the present invention is shown and will now be described. The primary differences between the cell culture flask 10d of this embodiments and the cell culture flask 10 of the previously described embodiment is that the flask body 12d includes a circumferential faceted portion 70 rather than the circumferential beveled portion 40. In that regard, the circumferential faceted portion 70 extends radially upwardly and outwardly between the base 20d of the flask body 12d and the outermost periphery 42 of the flask body 12d. The circumferential faceted portion 70 is defined by a plurality of facets 72 distributed circumferentially about flask body 12d. As described in further detail below, the circumferential faceted portion 70 provides sufficient turbulence needed to increase the oxygen exchange during agitation of the cell culture medium contained in the cell culture flask 10d to effectively culture certain cell types sensitive to shear stress.

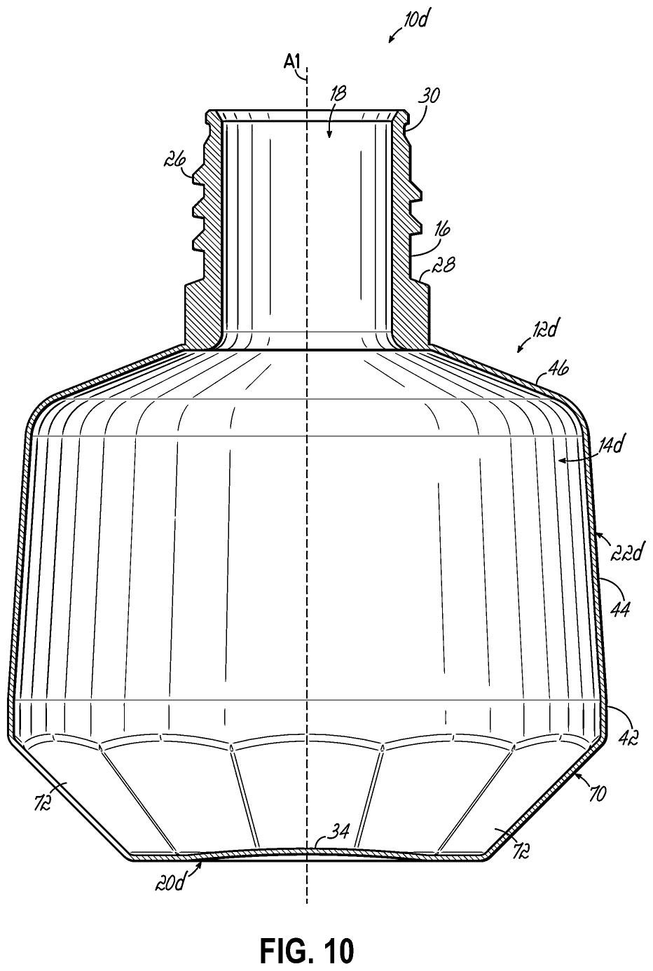

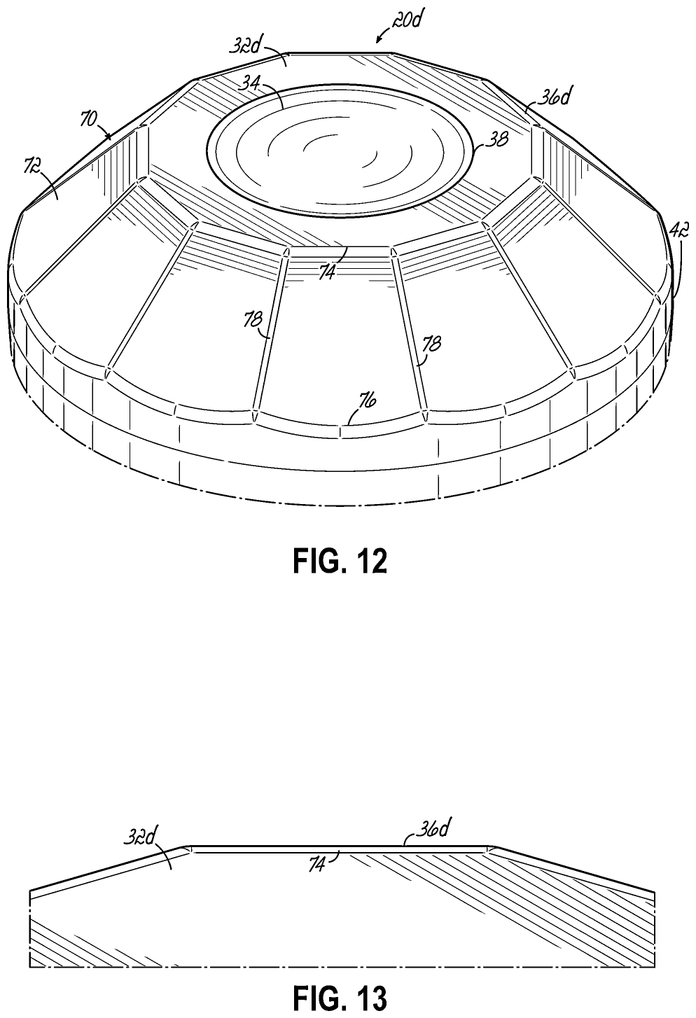

With reference to FIGS. 9-10, the cell culture flask 10d includes a flask body 12d that defines a cavity 14d for receiving a cell culture medium for culturing one or more cells, and an elongate neck 16 that defines an opening 18 to the cavity 14d. The flask body 12d further includes a base 20d and a sidewall 22d that extends from the elongate neck 16 to the base 20d. As best shown in FIG. 12, the base 20d of the flask body 12d includes a base surface 32d and a central concave portion 34 that is curved gently upward and into the cavity 14d from the base surface 32d. To this end, the base surface 32d comprises a generally flat annular disk having an outer circumferential edge 36d that defines an outer diameter of the base surface 32d and an inner circumferential edge 38 that defines an inner diameter of the base surface 32d. As shown in FIG. 10, the sidewall 22d includes the circumferential faceted portion 70 and a second circumferential beveled portion 44 that extends from the outermost periphery 42 of the flask body 12d to a shoulder 46 of the flask body 12d.

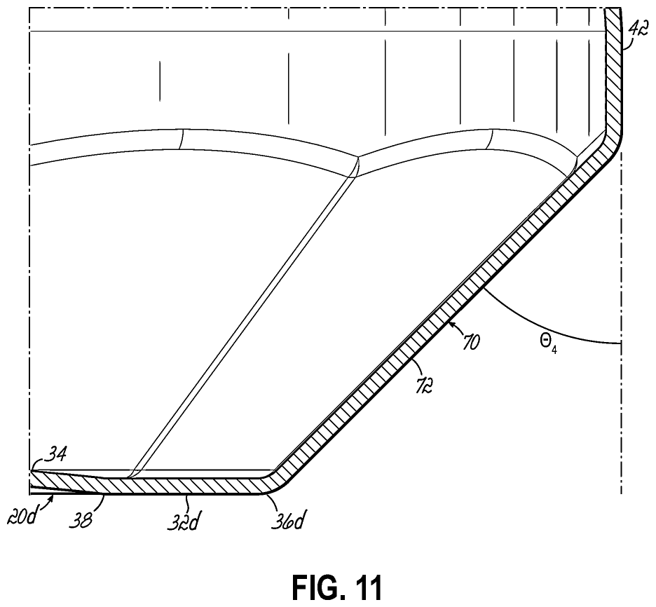

With reference to FIGS. 10 and 11, the circumferential faceted portion 70 extends upwardly from the base 20d of the flask body 12 to the outermost periphery 42 of the flask body 12d in a radially outwardly direction relative to the central axis A1 of the cell culture flask 10d. In particular, an angle of the circumferential faceted portion 70 is defined by an angle of each facet 72 relative to the central axis A1 (i.e., a vertical axis) of the cell culture flask 10d. As shown in FIG. 11, each facet 72 is angled at a constant angle relative to the central axis A1 of the cell culture flask 10d to define a facet angle θ₄. The facet angle θ₄ may be within a range of between 30° to 60° (or 30° to 60° from a horizontal base plane), for example. In a preferred embodiment, the facet angle θ₄ is 45°. To this end, the facets 72 provide a flat surface to support the cell culture flask 10d at an angle (equal to the facet angle θ₄) relative to a support surface on which the cell culture flask 10d sits to facilitate the recovery of cells from the cell culture flask 10d.

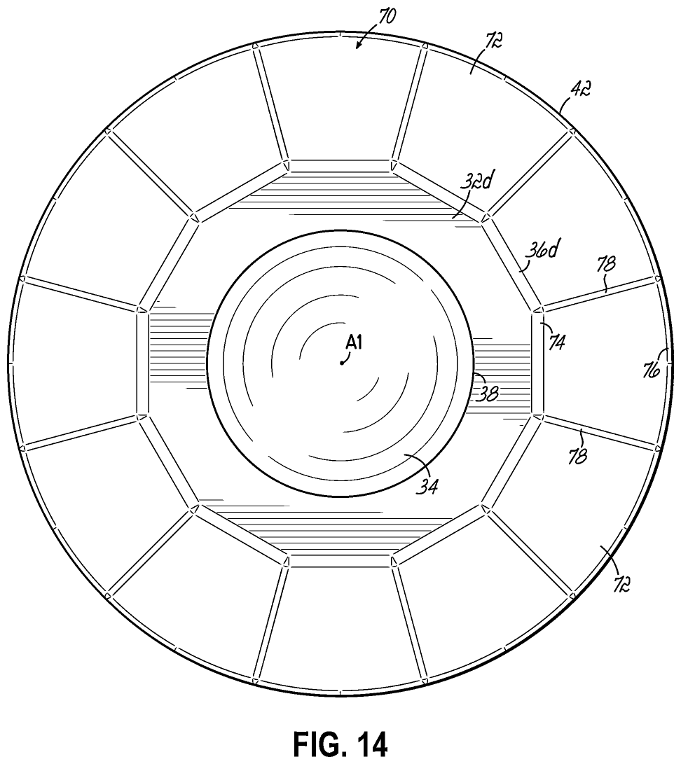

With reference to FIG. 12-14, the cell culture flask 10d includes 12 facets 72 arranged symmetrically about the central axis A1 of the cell culture flask 10d (e.g., FIG. 14). The 12-facet 72 configuration is particularly suitable for culturing cells sensitive to shear stress, however, it is possible that the cell culture flask 10d include fewer or more facets 72, as desired. As best shown in FIG. 12, each facet 72 defines a base edge 74 along the outer circumferential edge 36d of the base surface 32d, a rounded edge 76 at the outermost periphery 42 of the flask body 12d, and a pair of side edges 78 that extend between the base edge 74 and the rounded edge 76. Collectively, the facet base edges 74 define the outer circumferential edge 36d of the base surface 32d. In any event, agitation of the cell culture flask 10d with a laboratory shaker causes a vortex of the cell culture medium to flow over the plurality of facets 72, and in particular the side edges 78. To this end, the side edges 78 form a low point, or valley between each facet 72 that creates turbulent flow during agitation of the cell culture flask 10d by a laboratory shaker. Compared to a cell culture flask having baffles, the faceted only cell culture flask 10d greatly reduces the risk of foaming.

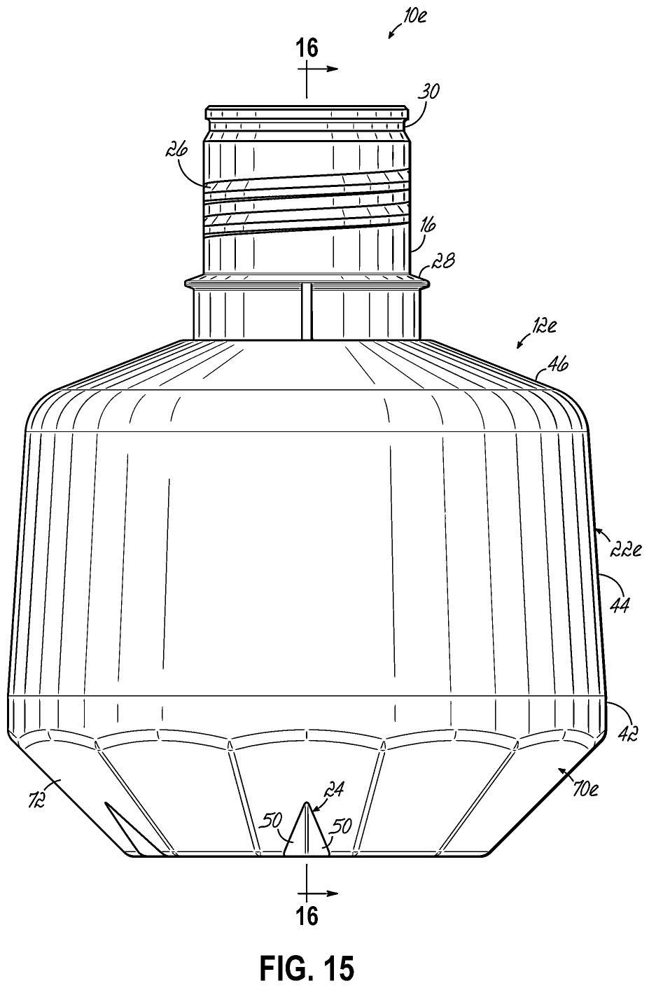

Referring now to FIGS. 15-21A, wherein like numerals represent like features compared to the embodiment of the cell culture flask 10d described above with respect to FIGS. 9-14, a cell culture flask 10e in accordance with another embodiment of the present invention is shown and will now be described. The primary differences between the cell culture flask 10e of this embodiments and the cell culture flask 10d of the previously described embodiment is that the cell culture flask 10e includes at least one baffle 24 formed in the flask body 12e that is configured to increase oxygen exchange during agitation of the cell culture medium contained in the cell culture flask 10e, particularly for cell types sensitive to shear stress, to thereby increase cell titer and protein expression while minimizing cell shear and foaming.

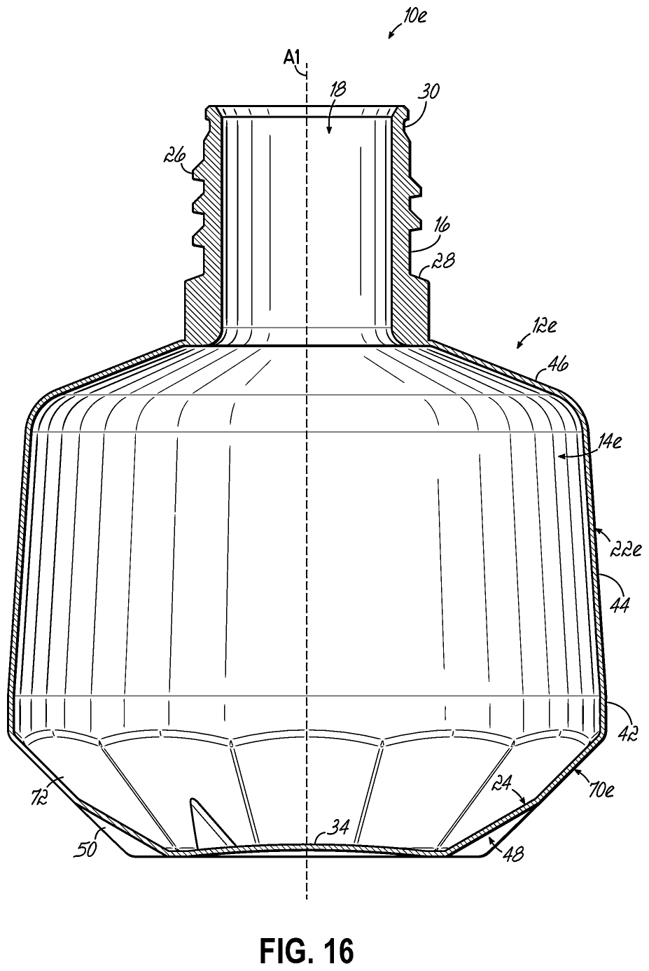

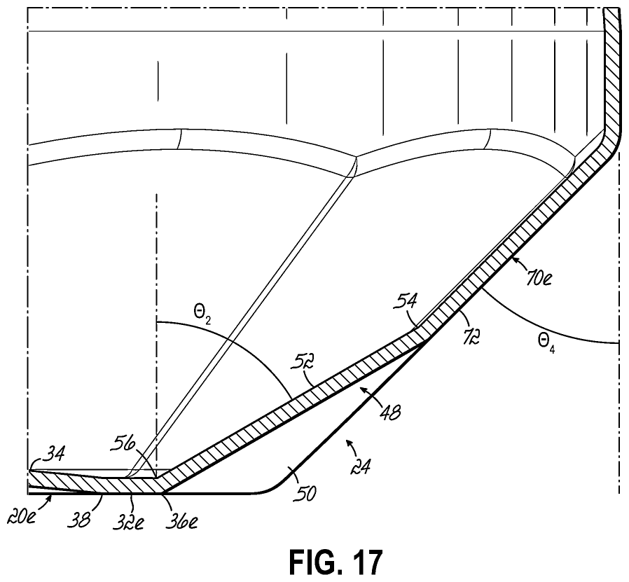

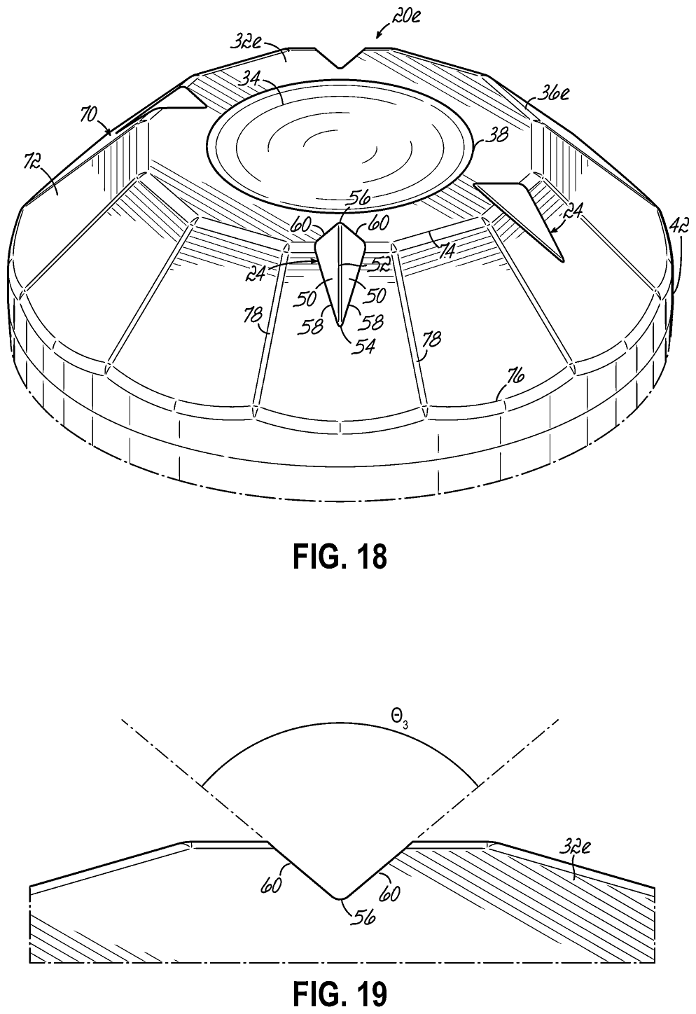

FIGS. 15-20 illustrate the cell culture flask 10e with four “small” baffles 24 formed in the flask body 12e, like the embodiment of the cell culture flask 10 described above with respect to FIGS. 1-7A. As shown in FIGS. 16 and 17, the pair of sidewalls 50 of each baffle 24 extend between the circumferential faceted portion 70e of the sidewall 22e and the base surface 32e of the flask body 12e such that the edge 52 extends in a radially inward direction relative to the central axis A1 of the cell culture flask 10e. In that regard, the edge 52 extends from a first terminal end 54 located on a facet 72 of the circumferential faceted portion 70e of the sidewall 22e and a second terminal end 56 located on the base surface 32e of the flask body 12e. The first terminal end 54 of the edge 52 may be generally centered between side edges 78 of respective facet 72, as shown in FIG. 18. As shown in FIG. 17, the edge 52 of each baffle 24 is angled relative to the central axis (i.e., a vertical axis) of the cell culture flask 10 to define a baffle angle θ₂. The baffle angle θ₂ may be within a range of between 45° to 70° (or 20° to 45° from a horizontal base plane), for example. In a preferred embodiment, the baffle angle θ₂ is 60° (or 30° from a horizontal base plane).

With reference to FIGS. 18 and 19, each baffle 24 is generally V-shaped in transverse cross-section as a result of the angled relationship between the pair of sidewalls 50. More particularly, as shown in FIG. 19, the pair of sidewalls 50 are angled relative to each other to form a baffle sidewall angle θ₃ therebetween. The baffle sidewall angle θ₃ may be within a range of between 90° to 110°, for example. In a preferred embodiment, the baffle angle θ₃ is 100°.

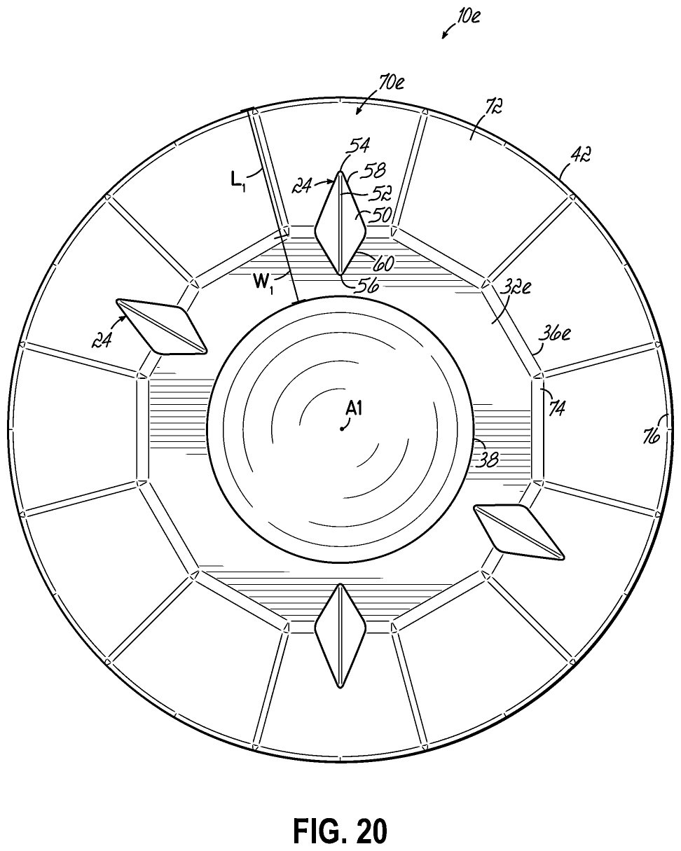

Referring now to FIG. 20, the circumferential faceted portion of the sidewall, and in particular each facet 72, includes a length L₁ measured as a distance between base edge 74 and the rounded edge 76 of each facet 72. Each first leg edge 58 may extend a distance from the base edge 74 (or outer edge 36e of the base surface 32e) to locate the first terminal end 54 of the edge 52 within an area on a facet 72 that is between 40% to 60%, and preferably 45% to 50%, of the length L₁ of the facet 72 measured from base edge 74 of the facet 72. Similarly, the base surface 32e includes a width W₁ measured as a distance between the inner circumferential edge 38 and the outer edge 36e of the base surface 32e of the flask body 12e. In that regard, each second leg edge 60 may extend a distance from the outer edge 36e of the base surface 32e (or the base edge 74 of the face 72) to locate the second terminal end 56 of the edge 52 within an area on the base surface 32e that is between 50% to 70%, and preferably 60% to 65%, of the width W₁ of the base surface 32e measured from the outer edge 36e of the base surface 32e of the flask body 12e.

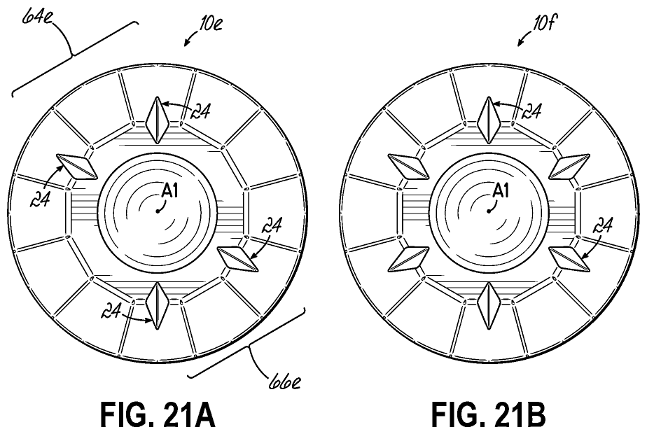

With reference to FIG. 21A, the cell culture flask 10e includes four baffles 24 with a first pair 64e, or grouping of baffles 24 and a second pair 66e, or grouping of baffles 24 diametrically opposed from the first pair 64e of baffles 24 about the central axis A1 of the cell culture flask 10e. The first pair 64e of baffles 24 are spaced 60° apart from each other about the central axis A1 of the cell culture flask 10e and the second pair 66e of baffles 24 are spaced 60° apart from each other about the central axis A1 of the cell culture flask 10e.

FIG. 21B illustrates a cell culture flask 10f having six baffles 24 in accordance with another embodiment of the present invention. As shown, the baffles 24 are spaced apart in a symmetrical arrangement about the central axis A1 of the cell culture flask 10f with the baffles 24 being spaced equidistantly apart in 60° increments about the central axis A1 of the cell culture flask 10f. To this end, the cell culture flask 10f may include fewer or more baffles 24 spaced apart in different increments about the central axis A1 of the cell culture flask 10f, such as four baffles 24 spaced 90° apart about the central axis A1 of the cell culture flask 10f, for example.

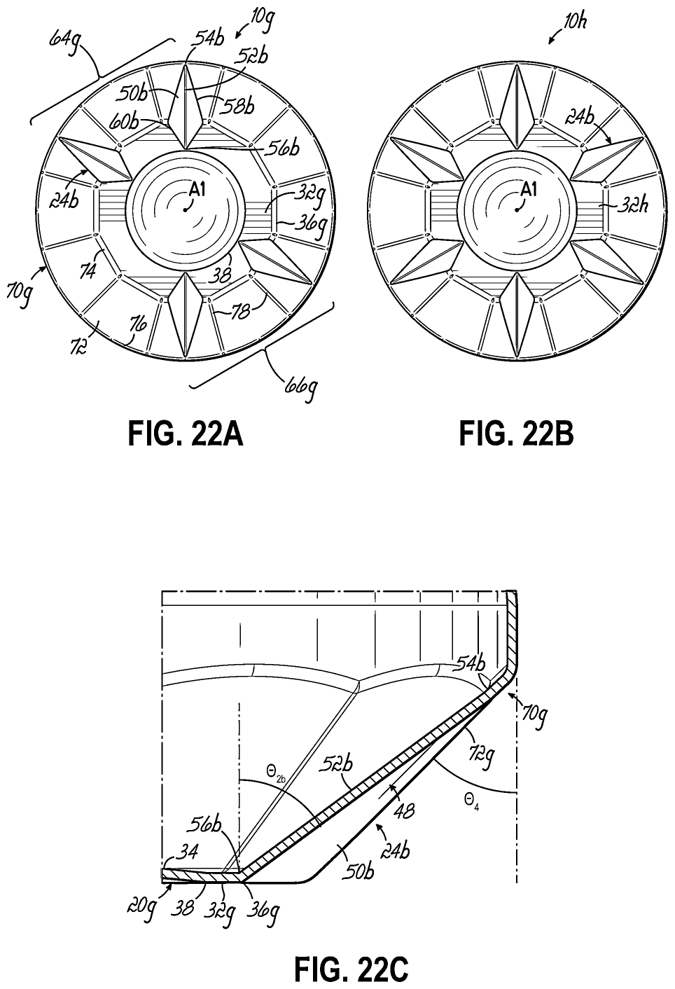

Referring now to FIGS. 22A-22C, wherein like numerals represent like features compared to the embodiment of the cell culture flask 10e described above with respect to FIGS. 15-21A, cell culture flasks 10g, 10h in accordance with additional embodiments of the present invention are shown and will now be described. The primary difference between the cell culture flask 10g, 10h of these embodiments and the cell culture flask 10e of the embodiment described above is the use of large baffles 24b, like the embodiment of the cell culture flask 10b described above with respect to FIGS. 8A-8C. As shown, each first leg edge 58b extends a distance from the base edge 74 of the facet 72 (or the outer edge 36g of the base surface 32g) to locate the first terminal end 54b of the edge 52b within an area on each facet 72 that is between 85% to 99%, and preferably 95% to 99%, of the length L₁ of the facet 72 measured from the base edge 74 of the facet 72. Similarly, each second leg edge 60b extends a distance from the base edge 74 of the facet 72 (or the outer edge 36g of the base surface 32g) to locate the second terminal end 56b of the edge 52b within an area on the base surface 32g that is between 85% to 99%, and preferably 95% to 99%, of the width of the base surface 32g measured from the base edge 74 of the facet 72. To this end, the baffle angle θ₂b is 52° (or 38° from a horizontal base plane), as shown in FIG. 22C.

With reference to FIG. 22A, the baffles 24b are spaced apart in a symmetrical arrangement about the central axis A1 of the cell culture flask 10g. In that regard, the cell culture flask 10g includes a first pair 64g, or grouping of large baffles 24b and a second pair 66g, or grouping of large baffles 24b diametrically opposed from the first pair 64g of baffles 24b about the central axis A1 of the cell culture flask 10g. The first pair 64g of baffles 24b are spaced 60° apart from each other about the central axis A1 of the cell culture flask 10g and the second pair 66g of baffles 24b are spaced 60° apart from each other about the central axis A1 of the cell culture flask 10g. To this end, each baffle 24b is formed on surfaces of a different one of the plurality of facets 72 and the base 32g.

FIG. 22B illustrates a cell culture flask 10h having six large baffles 24b according to another embodiment of the present invention, with the baffles 24b being spaced apart in a symmetrical arrangement about the central axis A1 of the cell culture flask 10h. In particular, the baffles 24b are spaced equidistantly apart in 60° increments about the central axis A1 of the cell culture flask 10h. To this end, each baffle 24b is formed on surfaces of a different one of the plurality of facets 72 and the base 32h.

The above-described cell culture flasks 10-10i may be molded using a blow molding manufacturing process, and may be formed from polyethylene terephthalate glycol (PETG). However, the cell culture flasks 10-10i may be formed using other manufacturing techniques, such as using a three-dimensional (3D) printing machine to print the cell culture flask 10-10i from a digital model, for example. Further, the cell culture flasks 10-10i may be molded from a variety of clear plastics which can be sterilized by radiation such as polycarbonate (PC), polystyrene (PS), polyethylene terephthalate (PET), assorted acrylic-based polymers (ACR), polymethylpentene (PMP), or any other suitable plastic that is optically clear to permit quick visual observations of the cell growth medium and cells such as for fill volume, foaming, media deterioration and contamination, pH shift, etc. The sidewall 22-22i of each cell culture flask 10-10i may be formed with volume graduations that are legible in specific units (e.g., metric units) from the outside of the cell culture flask 10a-10h when empty. For a 5 L cell culture flask, the volume graduations may be 0.5 L to 5 L in 0.5 L increments, for example.

FIG. 23 depicts an exemplary laboratory shaker 80 located within an incubator 82 for agitating and culturing cells located within an exemplary cell culture flask 10. However, the cell culture flask 10 may be any one of the embodiments 10-10i described above. The incubator 82 may be a Thermo Fisher Reach-In CO2 incubator model 3950 and the laboratory shaker may be a Thermo Fisher MAX Q16HP, a Thermo Scientific MaxQ 416HP, or a Thermo Scientific MaxQ 2000, each of which is commercially available from the Assignee of the present invention. As shown, the cell culture flask 10 contains an amount of fluid 84 (i.e., cell growth medium and cells) and the laboratory shaker 80 is configured to orbitally agitate the cell culture flask 10 at different agitation frequencies up to 150 rpm, for example, to express a protein of interest into the cavity 14 of the cell culture flaks 10. To this end, the orbital agitation of the cell culture flask 10 causes the liquid 84 held in the cell culture flask 10 (i.e., cell culture medium and cells) to swirl and flow up the sidewall 22 of the cell culture flask 10.

The present invention also contemplates a method of culturing cells using the above-described cell culture flasks 10-10i. The specific cell lines targeted and tested for culturing in certain embodiments of the cell culture flasks 10-10i described above are the following: Expi293 suspension-adapted human embryonic kidney (HEK) for growth in Gibco Expi293 Expression Medium; ExpiSf™ non-engineered derivative of Sf9 insect cells adapted for growth in ExpiSf™ CD Medium; and ExpiCHO derived from a non-engineered sub clone that has been screened and isolated from CHO—S Chinese hamster ovary (CHO) cells. However, the cell culture flask can be used to culture prokaryotic cells, mammalian cells such as a CHO cell, yeast cells, or insect cells, for example. With reference to FIG. 23, the method includes providing a cell culture flask 10-10i and introducing a volume of cell culture medium (e.g., Gibco Expi293 Expression Medium) and an amount of cells (e.g., Expi293) into the cavity 14 of the cell culture flask 10. Culturing the cells under conditions to support growth and/or expansion of the one or more cells, including agitating the cell culture flask with the laboratory shaker 80 at an agitation frequency of between 50 rpm to 150 rpm, for example, to express a protein of interest into the cavity 14. The method also includes isolating the protein of interest from the cell culture medium and/or the one or more cells.

In some embodiments, provided are methods of expressing a protein of interest by culturing cells configured to express the protein of interest using the above-described cell culture flasks 10-10i. under conditions to support expression of the protein of interest. In some embodiments, the protein of interest may include a recombinant protein. In some embodiments, the protein of interest includes a viral protein, such as without limitation a lentiviral protein or an adeno-associated viral protein. In some embodiments, the method further includes isolating the protein of interest from the cell culture medium and/or the one or more cells. Exemplary protein expression systems for use with the cell culture flasks provided herein include, without limitation, ExpiCHO™ Expression System, FreeStyle™ MAX CHO Expression System, Expi293™ Expression System, FreeStyle™ 293 Expression System, FreeStyle™ MAX 293 Expression System, and ExpiSf™ Expression System (all from Thermo Fisher Scientific).

In some embodiments, provided herein are methods of producing viral particles by culturing cells in a flask described herein under conditions to support virus particle production from the cells. In some embodiments, the cells are transfected with a recombinant viral vector, including without limitation a lentivirus vector or an adeno-associate virus vector, prior to culturing in the flask. In some embodiments, the cells are infected with a recombinant virus particle, including without limitation a lentivirus particle or an adeno-associate virus particle, prior to culturing in the flask. In some embodiments, the method further includes isolating the virus particles from the cell culture medium and/or cells following the culture period. Exemplary virus production systems for use with the cell culture flasks provided herein include, without limitation, AAV-MAX Helper-Free AAV Production System (Thermo Fisher Scientific) and LV-MAX™ Lentiviral Production System (Thermo Fisher Scientific).

### EXAMPLES

The following examples illustrate certain specific embodiments of the invention and are not meant to limit the scope of the invention. Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

Cell growth performance was assessed for cultures of several different cell lines grown using exemplary 5-liter beveled bottom, non-baffled flasks and 5-liter faceted bottom, non-baffled flasks as provided herein. These flasks are referred to in this Example as “beveled bottom flasks” and “faceted bottom flasks”, respectively.

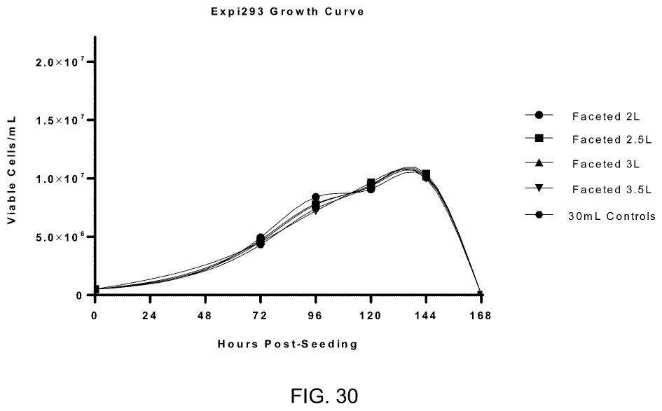

Expi293™ cells, suspension-adapted human embryonic kidney cell line, were seeded at a density of 0.5 million cells per mL in Expi293™ Expression Medium (Thermo Fisher Scientific) and grown in standard culture conditions. Starting from this very low cell density and doubling every 24 hours, the culture typically will reach a peak density after about 6 days in culture. Expi293 cell growth and viability was evaluated in culture volumes ranging from 2 liters up to 3.5 liters in the 5-liter flasks. An example of Expi293 cell growth performance in the faceted bottom flasks is shown in FIG. 30 with viable cells exceeding 1.0×107 cells/ml by day 6 post-seeding. The Expi293 cell growth performance in the 2 to 3.5 liter cultures was similar to that of the cells in a 30 ml culture volume.

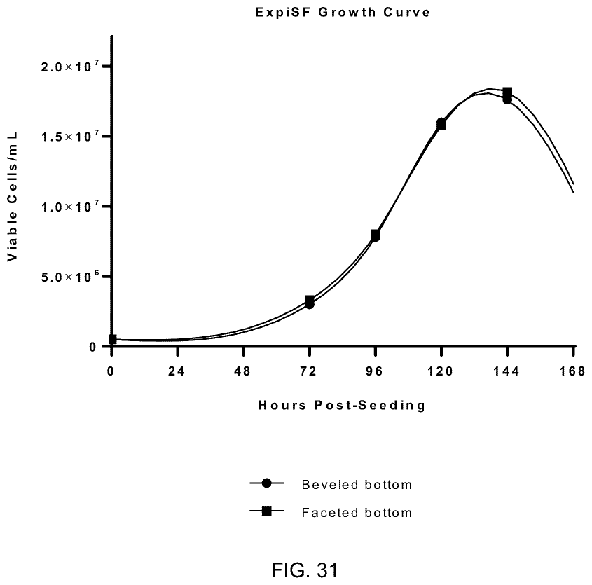

ExpiSf9™ cells, insect cells adapted for high-density suspension growth, were seeded at a density of 0.5 million cells per mL in ExpiSf9™ CD Medium (Thermo Fisher Scientific) and grown in standard culture conditions per manufacture's recommendations. Starting from this very low cell density and doubling every 24 hours, the culture typically will reach a peak density after about 6 days in culture. ExpiSf9 cell growth and viability was evaluated in culture volumes ranging from 2 liters up to 3.5 liters in the 5-liter flasks. An example of ExpiSf9 cell growth performance in 2-liter cultures in the beveled bottom flask and the faceted bottom flask is shown in FIG. 31 with viable cells reaching peak density of about 1.8×107 cells/ml by about day 6 post-seeding.

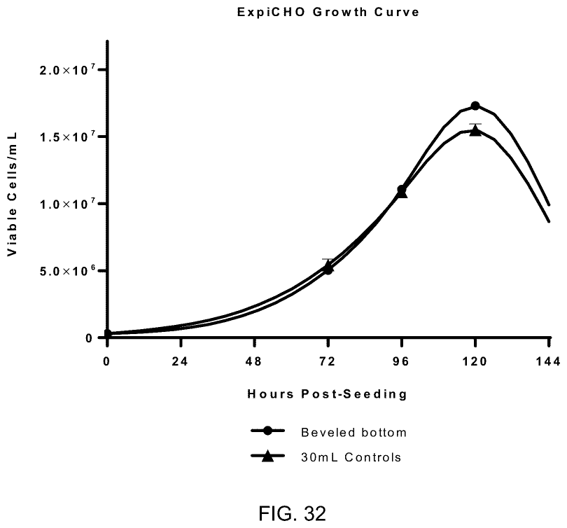

ExpiCHO™ cells, Chinese hamster ovary cells doe high density suspension culture, were seeded at a density of 0.3 million cells per mL in ExpiCHO™ Expression medium (Thermo Fisher Scientific) and grown in standard culture conditions. Starting from this very low cell density and doubling every 18-20 hours, the culture typically will reach a peak density after 5 days post-seeding. ExpiCHO cell growth and viability was evaluated in culture volumes ranging from 2 liters up to 3.5 liters in the 5-liter faceted bottom flasks. An example of ExpiCHO cell growth performance of a 2-liter culture in the beveled-bottom flask is shown in FIG. 32 with viable cells reaching peak density of about 1.7×10⁷ cells/ml by about day 6 post-seeding.

### Example 2

Recombinant protein expression from several cell types was assessed for cultures using exemplary 5-liter beveled bottom, non-baffled flasks and 5-liter faceted bottom, non-baffled flasks as provided herein. These flasks are referred to in this Example as “beveled bottom flasks” and “faceted bottom flasks”, respectively. Protein yield (titer) from the large culture volume flasks was compared to that of small-scale control flasks of 30 ml end volume.

Using the Expi293™ Expression System (Thermo Fisher Scientific), Expi293™ cells were seeded at a density of 3×10⁶ cells/mL of culture in Expi293™ Expression medium and transfected with an antibody expression vector plasmid DNA per manufacture instructions. The following day Expi293™ Transfection Enhancers were added to the transfected cell culture according to manufacture instructions. After 6 days of culture, the titer of antibody expressed and secreted into the culture medium was determined.

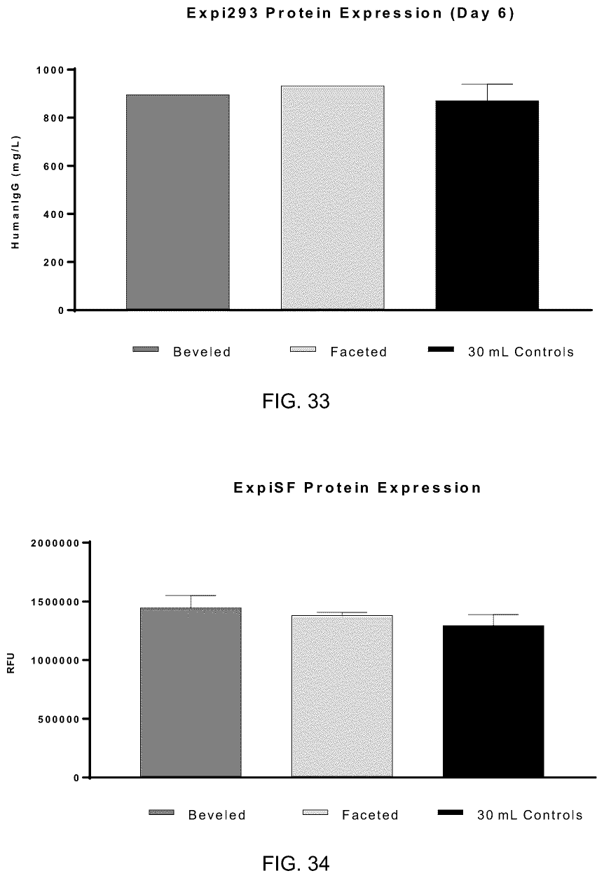

Recombinant protein production was evaluated in Expi293 culture volumes ranging from 2 liters to 2.5 liters in the 5-liter beveled-bottom and faceted-bottom flasks. Protein production results from the large volume cultures were comparable to that of the small-scale control flasks. An example of recombinant antibody production in a 2.25-liter end volume Expi293 culture is shown in FIG. 33.

To assess protein production using the ExpiSf9™ cells, recombinant baculovirus containing GFP-encoding DNA was prepared using the Bac-to-Bac™ Baculovirus Expression System (Thermo Fisher Scientific). Using the ExpiSf™ Expression System (Thermo Fisher Scientific), ExpiSf9™ cells were seeded at a density of 5×10⁶ cells/mL of culture in ExpiSf™ CD medium and ExpiSf™ Enhancer added to the culture per manufacturer instructions. The following day, the cells were infected with a baculovirus stock containing GFP-encoding DNA. After 3 days, the culture was harvested and GFP protein titer was quantified using a plate reader.

Recombinant protein production was evaluated in ExpiSf9 culture volumes ranging from 2 liters to 2.5 liters in the 5-liter beveled-bottom and faceted-bottom flasks. Protein production results from the large volume cultures were comparable to that of the small-scale control flasks. An example of GFP production in a 2-liter end volume ExpiSf9 culture is shown in FIG. 34.

Using the ExpiCHO™ Expression System (Thermo Fisher Scientific), ExpiCHO™ cells were seeded at a density of 6×10⁶ cells/mL of culture in ExpiCHO™ Expression medium and transfected with an antibody expression vector plasmid DNA per manufacture instructions. The following day ExpiCHO™ Enhancer and ExpiCHO™ Feed were added to the transfected cell culture according to manufacture instructions. After 8-14 days of culture, the titer of antibody expressed and secreted into the culture medium was determined.

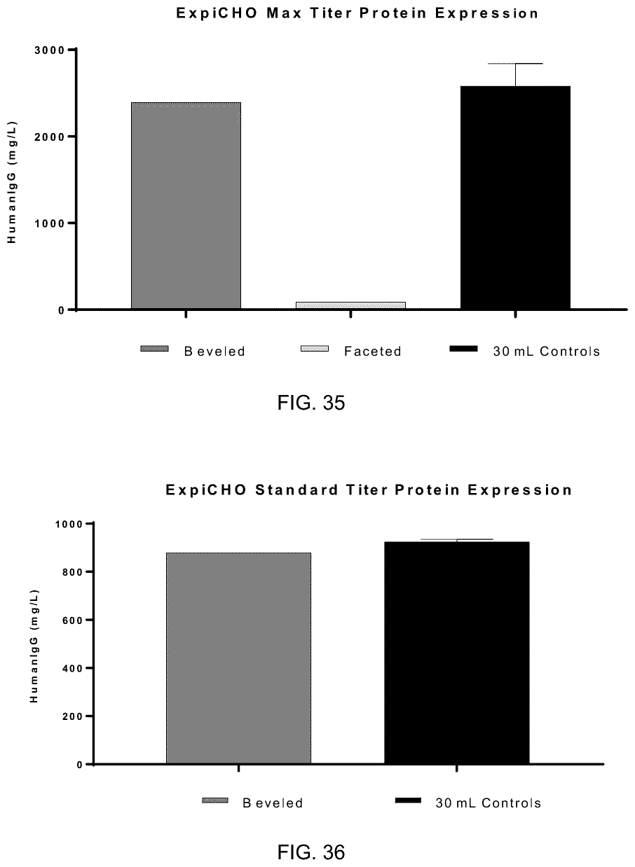

When the manufacturer's ExpiCHO™ Max Titer protocol was followed, the incubation temperature was decreased from 36.5° C. to 32° C. at one day after transfection, another volume of ExpiCHO™ Feed was again to the culture at day 5 post-transfection, and the cells were harvested at day 14. An example of recombinant antibody production in a 2-liter end volume ExpiCHO culture following the Max Titer protocol is shown in FIG. 35. An example of recombinant antibody production in a 2-liter end volume ExpiCHO culture following a variation of the Standard protocol is shown in FIG. 36. With the beveled-bottom flasks, protein production results from large volume cultures of transfected cells was similar to that of the small-scale control flasks. Surprisingly, the lack of success of protein production following CHO cell transfection in large volumes observed using other culture vessels was overcome with the use of beveled-bottom flasks provided herein.

While the invention has been illustrated by the description of various embodiments thereof, and while the embodiments have been described in considerable detail, it is not intended to restrict or in any way limit the scope of the appended claims to such detail. Thus, the various features discussed herein may be used alone or in any combination. Additional advantages and modifications will readily appear to those skilled in the art. The invention in its broader aspects is therefore not limited to the specific details and illustrative examples shown and described. Accordingly, departures may be made from such details without departing from the scope of the general inventive concept.

1. A cell culture flask, comprising:
a flask body defining a cavity for receiving a cell culture medium and comprising:
an elongate neck that defines an opening to the cavity;
a base;
a sidewall that extends from the elongate neck to the base of the flask body, the sidewall including a circumferential beveled portion that extends from an outermost periphery of the flask body to the base of the flask body at a constant angle relative to a central axis of the flask to define a sidewall angle.

2. The cell culture flask of claim 1, wherein the sidewall angle is within a range of between 30° to 45°.

3. The cell culture flask of claim 1, wherein the base includes a base surface having a width measured as a distance between an outer circumferential edge of the base surface and an inner circumferential edge of the base surface.

4. The cell culture flask of claim 3, wherein the base surface comprises a central concave portion.

5. The cell culture flask of claim 1, the flask further comprising at least one baffle formed in the flask body so as to extend in a radially inward direction relative to the central axis of the flask to form an indent in the flask.

6-20. (canceled)

21. The cell culture flask of claim 5, wherein the at least one baffle includes:
(i) a first pair of baffles spaced 60° apart from each other about the central axis of the flask and a second pair of baffles spaced 60° apart from each other about the central axis of the flask, wherein the first pair of baffles are diametrically opposed from the second pair of baffles about the central axis of the flask; or
(ii) six baffles spaced equidistantly apart and distributed circumferentially about the flask body.

22. (canceled)

23. A cell culture flask, comprising:
a flask body defining a cavity for receiving a cell culture medium and comprising:
an elongate neck that defines an opening to the cavity;
a base;
a sidewall that extends from the elongate neck to the base of the flask body, the sidewall including a circumferential faceted portion that extends from an outermost periphery of the flask body to the base of the flask body that is defined by a plurality of facets distributed circumferentially about the flask body.

24. The cell culture flask of claim 23, wherein each of the plurality of facets extends from the outermost periphery of the flask body to the base of the flask body at a constant angle relative to the central axis of the flask to define a facet angle.

25. The cell culture flask of claim 24, wherein each facet angle is within a range of between 30° to 60°.

26. The cell culture flask of claim 23, wherein the base includes a base surface having an outer circumferential edge that defines an outer diameter of the base surface and an inner circumferential edge that defines an inner diameter of the base surface.

27. The cell culture flask of claim 26, wherein the base surface comprises a central concave portion.

28. The cell culture flask of claim 23, the flask further comprising at least one baffle formed in surfaces of one facet of the plurality of facets and the base of the flask body so as to extend in a radially inward direction relative to a central axis of the flask.

29-44. (canceled)

45. The cell culture flask of claim 28, wherein the at least one baffle includes:
(i) a first pair of baffles spaced 60° apart from each other about the central axis of the flask so as to be formed on surfaces of a different one of the plurality of facets and the base, and a second pair of baffles spaced 60° apart from each other about the central axis of the flask so as to be formed on surfaces of a different one of the plurality of facets and the base, wherein the first pair of baffles are diametrically opposed from the second pair of baffles about the central axis of the flask; or
(ii) six baffles spaced equidistantly apart and distributed circumferentially about the flask body with each baffle being formed on surfaces of a different one of the plurality of facets and the base.

46. (canceled)

47. A method of culturing cells, comprising:
providing a cell culture flask according to claim 1;
introducing the cell culture medium and one or more cells into the cavity; and
culturing the cells under conditions to support growth and/or expansion of the one or more cells.

48-49. (canceled)

50. The method of claim 47, wherein the one or more cells is a mammalian cell, optionally a human embryonic kidney (HEK) cell or a Chinese hamster ovary (CHO) cell.

51. (canceled)

52. A method of expressing a protein of interest, comprising:
providing a cell culture flask according to claim 1;
introducing a cell culture medium and one or more cells configured to express the protein of interest into the cavity;
culturing the cells under conditions to support expression of the protein of interest; and
optionally isolating the protein of interest from the cell culture medium and/or the one or more cells.

53-56. (canceled)

57. A method of producing a virus vector, comprising:
providing a cell culture flask according to claim 1;
introducing a cell culture medium and one or more cells configured to express the virus vector into the cavity;
culturing the cells under conditions to support expression of the virus vector; and
optionally isolating the virus vector from the cell culture medium and/or the one or more cells.

58-60. (canceled)

61. A method of culturing cells, comprising:
providing a cell culture flask according to claim 23;
introducing the cell culture medium and one or more cells into the cavity; and
culturing the cells under conditions to support growth and/or expansion of the one or more cells.

62. A method of expressing a protein of interest, comprising:
providing a cell culture flask according to claim 23;
introducing a cell culture medium and one or more cells configured to express the protein of interest into the cavity;
culturing the cells under conditions to support expression of the protein of interest; and
optionally isolating the protein of interest from the cell culture medium and/or the one or more cells.

63. A method of producing a virus vector, comprising:
providing a cell culture flask according to claim 23;
introducing a cell culture medium and one or more cells configured to express the virus vector into the cavity;
culturing the cells under conditions to support expression of the virus vector; and
optionally isolating the virus vector from the cell culture medium and/or the one or more cells.
